(19) United States
(12) Patent Application Publication
(10) Pub. No.: US 2025/0270330 A1
(43) Pub. Date: Aug. 28, 2025

(54) TREATMENT OF HEADACHE DISORDERS WITH NK3 MODULATORS

(71) Applicant: Kallyope, Inc., New York, NY (US)

(72) Inventors: Justine KUPFERMAN, New York, NY (US); Justin N.J. MCMANUS, New York, NY (US); John WHANG, New York, NY (US)

(21) Appl. No.: 18/859,693

(22) PCT Filed: Apr. 21, 2023
(86) PCT No.: PCT/US2023/019406

§ 371 Date: Oct. 24, 2024

Related U.S. Application Data
(60) Provisional application No. 63/334,308, filed on Apr. 25, 2022.

(51) Int. Cl. C07K 16/28; A61K 31/4545; A61K 31/47; A61K 31/519; A61K 31/5383; A61K 31/7105; A61P 25/06

(52) U.S. Cl. CPC C07K 16/286; A61K 31/4545; A61K 31/47; A61K 31/519; A61K 31/5383; A61K 31/7105; A61P 25/06; C07K 2319/00

(57) ABSTRACT

The present invention relates to a method for treating or preventing headache disorders selected from the group consisting of migraine, medication overuse headache, cluster headache, general headache, trigeminal neuralgia, and orofacial pain, comprising administering a therapeutically effective amount of a therapeutic agent that modulates activity of a Neurokinin Receptor 3 (“NK3”) protein or modulates expression of a TACR3 gene.

### CROSS-REFERENCE

This application claims the benefit of U.S. Provisional Application Ser. No. 63/334,308 filed Apr. 25, 2022, which is hereby incorporated by reference in its entirety.

### BACKGROUND OF THE INVENTION

Migraine is a painful and debilitating neurological condition of the trigeminovascular system with high prevalence in the general population, affecting approximately 15% of individuals, making it the second leading cause of disability worldwide. Migraine is characterized by recurrent, pulsating headache attacks of moderate to severe pain intensity with sensitivity to movement, visual, auditory, and other sensory input that can last from 4 to 72 hours.

While there are several classes of oral drugs approved for migraine treatment, many of those currently in use are associated with significant side effects, leading to frequent discontinuation and class switching along with poor outcomes. Antibody therapies and small molecules targeting CGRP receptors have fewer reported side effects, but less than a third of migraineurs are responsive to these treatments. Thus, there is significant need for novel therapies to treat migraine and other disorders of the trigeminovascular system such as cluster and medication overuse headache and trigeminal neuralgia.

### SUMMARY OF THE INVENTION

In some aspects, the present disclosure provides a method for treating or preventing a disease or condition in a subject in need thereof, the method comprising administering to the subject a therapeutically effective amount of a therapeutic agent that modulates activity of a Neurokinin Receptor 3 (“NK3”) protein or modulates expression of a TACR3 gene, wherein the disease or condition is selected from the group consisting of: migraine, cluster headache, general headache, medication overuse headache, trigeminal neuralgia, and orofacial pain.

In another aspect, the present disclosure provides an NK3 modulator for use in the treatment or prevention of a disease or condition selected from the group consisting of: migraine, cluster headache, general headache, medication overuse headache, trigeminal neuralgia, and orofacial pain, wherein the NK3 modulator is a therapeutic agent that modulates activity of an NK3 protein or modulates expression of a TACR3 gene.

In some embodiments, the disease or condition is selected from the group consisting of: migraine, medication overuse headache, cluster headache, and general headache. In some embodiments, the disease or condition is migraine.

In some embodiments, the therapeutic agent modulates activity of an NK3 protein. In some embodiments, the therapeutic agent is an NK3 antagonist. In some embodiments, the therapeutic agent modulates expression of a TACR3 gene. In some embodiments, the therapeutic agent decreases expression of a TACR3 gene.

In some embodiments, the administration of the therapeutic agent does not reduce luteinizing hormone in the subject (e.g., when the therapeutic agent is administered at the therapeutically effective dose). In some embodiments, the administration of the therapeutic agent results in a ratio of blood plasma concentration to brain tissue concentration of at least 10 to 1 in the subject. In some embodiments, the administration of the therapeutic agent results in a ratio of blood plasma concentration to brain tissue concentration of at least 100 to 1 in the subject. In some embodiments, the administration of the therapeutic agent selectively modulates activity of the NK3 protein or expression of the TACR3 gene in peripheral tissues compared to the central nervous system. In some embodiments, the administration of the therapeutic agent selectively modulates activity of the NK3 protein or expression of the TACR3 gene in the peripheral nervous system compared to the central nervous system.

In some embodiments, the therapeutic agent is a small molecule, a protein, or a nucleic acid. In some embodiments, the therapeutic agent is a small molecule.

In some embodiments, wherein the therapeutic agent is disclosed in EP0673928; WO1995032948; WO1997010211; WO1997019928; WO1997019927; WO1997021680; WO1998018785; WO1998057972; WO1999026924; WO1999001451; WO2000021931; WO2000064877; WO2000039114; WO2002013825; WO2002094821; WO2002083645; WO2002038547; WO2002038548; WO2002044154; WO2002043734; WO2002044165; WO2004066951; WO2004066950; WO2005014575; WO2005061462; WO2005016884; WO2005110987; US2005148601; WO2006050989; WO2006050991; WO2006050992; WO2006130080; WO2006137789; WO2007086799; WO2007018465; WO2007018469; WO200700396; WO2007035157; WO2007018466; WO2007035156; WO2007035158; WO2007069977; WO2007012900; WO2007028654; US20070219214; EP1829556; WO2008115140; WO2008131779; WO2008148688; WO2008148689; WO2008151969; WO2008081012; WO2009130240; WO2009156339; WO2009033995; WO2009019163; WO2009024502; WO2009072643; WO2010106081; WO2010045948; WO2010028655; WO2010086259; WO2010015626; WO2020101017; JP201081888; WO2011110183; WO2011121137; WO2012039718; WO2012009227; WO2012004207; WO2013050424; WO2014154895; WO2014154896; WO2014089019; WO2015200594; WO2015033163; WO2019210327; WO2019074081; or WO2021030335.

In some embodiments, the small molecule is selected from fezolinetant, pavinetant, talnetant, osanetant, elinzanetant, senktide, GSK172981, GSK256471, SB222200, SB235375, SJX-653, MLE-301, SB218795, GR138676, GR138678, CAM-2425, SSR241586, SSR146977, PD154740, PD157672, PD163416, PD160946, PD161182, SCH206272, GSK256471, and SB48968. In some embodiments, the small molecule is selected from fezolinetant, pavinetant, talnetant, osanetant, elinzanetant, senktide, GSK172981, GSK256471, SB222200, SB235375, SJX-653, MLE-301, SB218795, GR138676, GR138678, CAM-2425, SSR241586, SSR146977, PD154740, PD157672, PD163416, PD160946, and PD161182. In some embodiments, the small molecule is selected from fezolinetant, pavinetant, talnetant, osanetant, elinzanetant, GSK172981, GSK256471, SB222200, SB235375, SJX-653, and MLE-301.

In some embodiments, the therapeutic agent is a protein. In some embodiments, the therapeutic agent is an antibody or a functional fragment thereof that selectively binds NK3 or neurokinin B. In some embodiments, the therapeutic agent is an antibody or a functional fragment thereof that selectively binds NK3 disclosed in WO2012020162A1. In some embodiments, the therapeutic agent is an antibody or a functional fragment thereof that selectively binds neurokinin B disclosed in WO2003102136 or WO2020101017. In some embodiments, the therapeutic agent is a fusion protein.

In some embodiments, the therapeutic agent is a nucleic acid. In some embodiments, the therapeutic agent is a RNAi molecule. In some embodiments, the RNAi molecule is a shRNA, a siRNA, a microRNA, or an asymmetric interfering RNA. In some embodiments, the therapeutic agent is an antisense molecule, a lhRNA, a miRNA embedded shRNA, or a small internally segmented RNA. In some embodiments, the therapeutic agent is administered via a viral vector.

In some embodiments, the subject is a human.

In another aspect, the present disclosure provides an NK3 modulator for use in the treatment or prevention of a disease or condition selected from the group consisting of: migraine, cluster headache, general headache, medication overuse headache, trigeminal neuralgia, and orofacial pain, wherein the NK3 modulator is a therapeutic agent that modulates activity of an NK3 protein or modulates expression of a TACR3 gene.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIGS. 1A-B show the effects of a brain penetrant NK3 antagonist, fezolinetant, (FIG. 1A) and a peripherally restricted NK3 antagonist, SB 235375, (FIG. 1B) on periorbital threshold following nitroglycerin (hereinafter “GTN”) exposure in an in vivo migraine headache model using rats with inflammatory soup-sensitized trigeminal ganglia.

FIG. 2 shows the effects of NK3 antagonists on luteinizing hormone (LH) levels in adult castrated rats at 30, 90, and 150 minutes post dosing with fezolinetant or SB 235375.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Neurokinin receptor 3 (“NK3”) (also known as tachykinin receptor 3) is encoded by the TACR3 gene (also referred to herein as “NK3 gene”). NK3 modulators are provided herein for the treatment of headache disorders and diseases, such as cluster headaches and migraines. Such NK3 modulators include both agents that modulate the activity of an NK3 protein (e.g., agents that bind to and/or modulate the activity of an NK3 protein), as well as agents that modulate the expression of the TACR3 gene (e.g., agents that modulate the level of NK3 protein by changing the expression level of a TACR3 gene, which encodes for the NK3 protein). The present disclosure demonstrates the efficacy of NK3 modulators in well-known migraine models. Further, in some embodiments of the present disclosure, the NK3 modulator is peripherally restricted (e.g. has little to no penetration into the central nervous system (“CNS”), such that the NK3 modulator only produces pharmacological effects on peripheral tissues (including, e.g., tissues of the peripheral nervous system). Such peripherally restricted NK3 modulators possess the additional advantage of minimizing unwanted side effects caused by modulation of NK3 in the CNS, such as unwanted changes in hormone levels, including luteinizing hormone (LH).

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

The term “about” or “approximately” as used herein when referring to a measurable value such as an amount or concentration and the like, is meant to encompass variations of 20%, 10%, 5%, 1%, 0.5%, or even 0.1% of the specified amount. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, up to 5-fold, or up to 2-fold, of a value. Where particular values can be described in the application and claims, unless otherwise stated the term “about” may be assumed to encompass the acceptable error range for the particular value. Also, where ranges, subranges, or both, of values, can be provided, the ranges or subranges can include the endpoints of the ranges or subranges.

Where values are described as ranges, it may be understood that such disclosure includes the disclosure of all possible sub-ranges within such ranges, as well as specific numerical values that fall within such ranges irrespective of whether a specific numerical value or specific sub-range is expressly stated.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

As used herein, the term “antibody or antigen binding fragment thereof” is intended to cover polyclonal antibodies, multiclonal antibodies, monoclonal antibodies, chimeric antibodies, humanized and primatized antibodies, human antibodies, recombinantly produced antibodies, intrabodies, multispecific antibodies, bispecific antibodies, monovalent antibodies, multivalent antibodies, anti-idiotypic antibodies, synthetic antibodies, including muteins and variants thereof; antibody fragments such as Fab fragments, F(ab′) fragments, single-chain FvFcs, single-chain Fvs; and derivatives thereof including Fc fusions and other modifications, and any other immunologically active molecule so long as they exhibit the desired biological activity (i.e., antigen association or binding). Moreover, the term further comprises all classes of antibodies (i.e. IgA, IgD, IgE, IgG, and IgM) and all isotypes (i.e., IgG1, IgG2, IgG3, IgG4, IgA1, and IgA2), as well as variations thereof unless otherwise dictated by context.

The term “salt” or “pharmaceutically acceptable salt” refers to salts derived from a variety of organic and inorganic counter ions well known in the art. Pharmaceutically acceptable acid addition salts can be formed with inorganic acids and organic acids. Pharmaceutically acceptable base addition salts can be formed with inorganic and organic bases.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

The term “preventing” is art-recognized, and when used in relation to a condition, such as headache, is well understood in the art, and includes administration of a composition which reduces the frequency of, or delays the onset of, symptoms of a medical condition in a subject relative to a subject which does not receive the composition. Thus, prevention of headache includes, for example, reducing the number of patients suffering from headaches in a population of patients receiving a prophylactic treatment relative to an untreated control population. Prevention of a headache condition also includes, for example, reducing the number of diagnoses of the headache condition in a treated population versus an untreated control population, and/or delaying the onset of symptoms of the headache condition in a treated population versus an untreated control population.

As used herein, the phrase “therapeutically effective amount,” means the amount of a compound that, when administered to a patient for treating a disease, is sufficient to treat the disease or condition. The “therapeutically effective amount” will vary depending on the compound, the disease and its severity and the age, weight, adsorption, distribution, metabolism and excretion etc., of the patient to be treated.

As used herein, “treatment” or “treating” refers to an approach for obtaining beneficial or desired results with respect to a disease, disorder, or medical condition including, but not limited to, a therapeutic benefit. In certain embodiments, treatment or treating involves administering a compound or composition disclosed herein to a subject. A therapeutic benefit may include the eradication or amelioration of the underlying disorder being treated.

As used herein, “RNAi” refers to interfering RNA or RNA interference. RNAi refers to a means of selective post-transcriptional gene silencing by destruction of specific mRNA by molecules that bind and inhibit the processing of mRNA, for example inhibit mRNA translation or result in mRNA degradation. As used herein, the term “RNAi” refers to any type of interfering RNA, including but are not limited to, siRNAi, shRNAi, endogenous microRNA, artificial microRNA, or asymmetric interfering RNA. For instance, it includes sequences previously identified as siRNA, regardless of the mechanism of down-stream processing of the RNA.

As used herein, “modulate” refers to alteration of the activity or the expression of target protein/gene in the presence of a composition, relative to the comparable conditions in the absence of the composition. As used herein, the term “modulate” can be up-regulation (e.g., activation or stimulation) or down-regulation (e.g., inhibition or suppression). For example, modulation may cause a change in cellular level of the target protein or a change in expression of the target gene, stability of protein, enzymatic modification (e.g., phosphorylation) of the target protein, binding characteristics (e.g., binding to a target transcription regulatory element), or any other biological, functional, or immunological properties of the target protein. The change in activity can arise from, for example, an increase or decrease in expression of the target gene, the stability of mRNA that encodes the target protein, translation efficiency, or from a change in other bioactivities of the target protein transcription factor (e.g., regulating expression of the target protein-responsive gene). The mode of action of a target gene/protein modulator can be direct, e.g., through binding to the target protein or to genes encoding the target protein. The change can also be indirect, e.g., through binding to and/or modifying (e.g., enzymatically) another molecule which otherwise modulates target gene/protein (e.g., a kinase that specifically phosphorylates target protein).

As used herein, the term “administer”, “administered”, “administration”, or “to administer” refers to the step of giving (i.e. administering) a pharmaceutical composition to a subject, or alternatively a subject receiving a pharmaceutical composition. The pharmaceutical compositions disclosed herein can be locally administered by various methods. For example, intramuscular, intradermal, subcutaneous administration, intrathecal administration, intraperitoneal administration, topical (transdermal), instillation, and implantation (for example, of a slow-release device such as polymeric implant or miniosmotic pump) can all be appropriate routes of administration.

As used herein, the term “nucleic acid” refers to a ribonucleic or deoxyribonucleic acid or analog thereof, including a nucleic acid analyte presented in any context; for example, siRNA or microRNA. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be synthesized as a single stranded molecule or expressed in a cell (in vitro or in vivo) using a synthetic gene. Nucleic acids may be obtained by chemical synthesis methods or by recombinant methods. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof.

The terms “subject,” “individual,” and “patient” may be used interchangeably and refer to humans, as well as non-human mammals (e.g., non-human primates, canines, equines, felines, porcines, bovines, ungulates, lagomorphs, rodents, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for, a pathology to be prophylactically or therapeutically treated with a therapeutic protein described herein.

The term “specificity,” as used herein, refers to the ability of a protein binding domain, in particular, an immunoglobulin or an immunoglobulin fragment, such as a nanobody, to bind preferentially to one antigen versus a different antigen, and does not necessarily imply high affinity.

### Treatment of Headache Disorders

Provided herein are methods for treating a disease or condition in a subject in need thereof, comprising administering to the subject a therapeutically effective amount of a therapeutic agent that modulates activity of a Neurokinin Receptor 3 (“NK3”) protein or modulates expression of a TACR3 gene. In some embodiments, the disease or condition is a headache disorder. In some embodiments, the disease or condition is selected from migraine, medication overuse headache, cluster headache, general headache, tension headache, caffeine headache, hormone headache, hemicrania continua, hypertension headache, rebound headache, post-traumatic headache, exertion headache, spinal headache, thunderclap headache, icepick headache, trigeminal neuralgia, orofacial pain (e.g., chronic orofacial pain), or a combination of two or more of these diseases or conditions. Non-limiting examples of said combinations include migraine and general headache, general headache and trigeminal neuralgia, and cluster headache and orofacial pain. In some embodiments, the disease or condition is selected from the group consisting of: migraine, medication overuse headache, cluster headache, general headache, trigeminal neuralgia, and orofacial pain. In further embodiments, the disease or condition is migraine. In some embodiments, the subject is a mammal. In some embodiments, the subject is a human.

In some embodiments, the therapeutic agents described herein are used in the preparation of medicaments for the prevention or treatment of diseases or conditions. In some embodiments, the therapeutic agents described herein are used in the preparation of medicaments for the prevention or treatment of headache disorders. In some embodiments, the therapeutic agents described herein are used in the preparation of medicaments for the prevention or treatment of a disease or condition selected from migraine, medication overuse headache, cluster headache, general headache, tension headache, caffeine headache, hormone headache, hemicrania continua, hypertension headache, rebound headache, post-traumatic headache, exertion headache, spinal headache, thunderclap headache, icepick headache, trigeminal neuralgia, orofacial pain, or a combination of two or more of these diseases or conditions.

Described herein in some embodiments are NK3 modulators for use in treating or preventing a disease or condition. Described herein in some embodiments are NK3 modulators for use in treating or preventing a headache disorder. In some embodiments, the present disclosure provides NK3 modulator for use in the treatment or prevention of a disease or condition selected from the group consisting of: migraine, medication overuse headache, cluster headache, general headache, trigeminal neuralgia, and orofacial pain, wherein the NK3 modulator is a therapeutic agent that modulates activity of an NK3 protein or modulates expression of a TACR3 gene

In some embodiments, compositions comprising a therapeutic agent of the present disclosure is administered for prophylactic and/or therapeutic treatments. In some embodiments, NK3 modulators are administered to a patient already suffering from a headache disorder, in an amount sufficient to cure or at least partially arrest one or more symptoms of the headache disorder. Amounts effective for this use will depend on the severity and course of the disease or condition, previous therapy, the patient's health status, weight, and response to the drugs, and the judgment of the treating physician.

In some embodiments, administration of a therapeutic agent of the present disclosure results in cessation or reduction of one or more symptoms of a headache disorder. In some embodiments, administration of a therapeutic agent of the present disclosure results in cessation or reduction of one or more symptoms of a disease or condition selected from migraine, medication overuse headache, cluster headache, general headache, trigeminal neuralgia, and orofacial pain. In some embodiments, administration of a therapeutic agent of the present disclosure results in cessation or reduction of one or more symptoms selected from pain, sensitivity to light, sensitivity to sound, sensitivity to smell, aura, nausea, vomiting, agitation, vertigo, lightheadedness, muscle stiffness, muscle spasm, swelling, numbness, scalp tenderness and nasal congestion. In some embodiments, administration of a therapeutic agent of the present disclosure results in cessation or reduction of one or more symptoms selected from pain, sensitivity to light, sensitivity to sound, nausea, and vomiting. In some embodiments, administration of a therapeutic agent of the present disclosure results in cessation or reduction of pain (e.g., pain associated with a headache disorder). In some embodiments, administration of a therapeutic agent of the present disclosure results in freedom from pain.

In prophylactic applications, compositions containing a therapeutic agent described herein are administered to a patient susceptible to or otherwise at risk of a disorder or condition (e.g. a headache disorder). Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician.

A therapeutic agent of the present disclosure may be administered to the subject using different administration routes, including oral, transmucosal, topical, transdermal, inhalation, intravenous, subcutaneous, intradermal, intramuscular, intra-articular, perineural, intraventricular, intravenous, intraperitoneal, intranasal, and intraocular.

NK3 signaling has distinct functions in the CNS, where its regulation of KNDy (kisspeptin, NKB, dynorphin) neurons in the hypothalamus regulates gonadotropin releasing hormone (GnRH) that ultimately controls pulsatile pituitary secretions of LH. See Uenoyama, Y., Nagae, M., Tsuchida, H., Inoue, N. & Tsukamura, H., Role of KNDy Neurons Expressing Kisspeptin, Neurokinin B, and Dynorphin A as a GnRH Pulse Generator Controlling Mammalian Reproduction. 12 FRONT. ENDOCRINOL. 724632 (2021); Depypere, H., Lademacher, C., Siddiqui, E. & Fraser, G. L. Fezolinetant in the treatment of vasomotor symptoms associated with menopause, 30 EXPERT OPIN. INVESTIG. DRUGS 681-694 (2021) (hereinafter “Depypere 2021”). To avoid unwanted side effects associated with affecting the secretions of these gonadotropins, in some embodiments, the therapeutic agent selectively modulates NK3 in peripheral tissues over NK3 in the CNS. In some embodiments, the therapeutic agent selectively modulates NK3 in the peripheral nervous system over NK3 in the CNS

In some embodiments, the therapeutic agent is a peripherally restricted NK3 modulator. In some embodiments, the therapeutic agent is a peripherally restricted NK3 modulator (e.g., does not accumulate to a clinically significant concentration in the brain). In some embodiments, the use of a peripherally restricted NK3 modulator minimizes or avoids unwanted side effects associated with NK3 modulation in the CNS (e.g., changes in gonadotropin levels such as LH). Such side effects include thermoregulatory dysfunction, change in bone mineral density, paresthesia, thrombophlebitis, and change in one or more gonadotropin levels. In some embodiments, administration of the NK3 modulators disclosed herein lack one or more of the following side effects: bone mineral density changes, paresthesia, thrombophlebitis, infertility, changes in sex drive, menstrual cycle abnormalities (e.g. delayed or blocked ovulation), changes in testosterone levels, testicular shrinkage, and changes in gonadotropin levels (e.g., luteinizing hormone). In some embodiments, administration of the NK3 modulators disclosed herein do not reduce LH levels in the subject.

In some embodiments, administration of the therapeutic agent does not reduce luteinizing hormone in the subject at the therapeutically effective amount. In some embodiments, administration of a therapeutically effective amount of the therapeutic agent reduces luteinizing hormone in the subject, for example, by less than 1%, less than 2%, less than 3%, less than 4%, less than 5%, less than 6%, less than 7%, less than 8%, less than 9%, less than 10%, less than 11%, less than 12%, less than 13%, less than 14%, less than 15%, less than 20%, less than 25%, less than 30%, less than 35%, less than 40%, less than 45%, or less than 50%. In some embodiments, administration of a therapeutically effective amount of the therapeutic agent reduces the concentration of luteinizing hormone a sample obtained from the subject, for example, by less than 1%, less than 2%, less than 3%, less than 4%, less than 5%, less than 6%, less than 7%, less than 8%, less than 9%, less than 10%, less than 11%, less than 12%, less than 13%, less than 14%, less than 15%, less than 20%, less than 25%, less than 30%, less than 35%, less than 40%, less than 45%, or less than 50%. In some embodiments, the administration of the therapeutic agent at the therapeutic level reduces luteinizing hormone in the subject by less than about 50%, less than about 45%, less than about 40%, less than about 35%, less than about 30%, less than about 25%, less than about 20%, less than about 15%, less than about 10%, less than about 9%, less than about 8, less than about 7%, less than about 6%, less than about 5%, less than about 4%, less than about 3%, less than about 2%, less than about 1%, or less than about 0.5%. In some embodiments, the sample comprises, for example, blood, blood plasma, urine, mucus, or saliva. The foregoing examples are merely exemplary and are not intended to limit the scope of the present disclosure.

### NK3 Modulators

In some embodiments, the therapeutic agent that modulates activity of a Neurokinin Receptor 3 (“NK3”) protein or modulates expression of a TACR3 gene. In some embodiments, the therapeutic agent modulates activity of an NK3 protein. In some embodiments, the therapeutic agent is an NK3 modulator. In some embodiments, the therapeutic agent is an NK3 antagonist. In some embodiments, the therapeutic agent is an NK3 agonist. In some embodiments, the therapeutic agent binds to the NK3 protein thereby preventing binding of endogenous ligands to the NK3 protein. The methods of the present disclosure comprise administration of a therapeutic agent that may modulate activity of an NK3 protein. The therapeutic agent may enhance or diminish NK3 protein activity. In some embodiments, at a therapeutically effective concentration, the therapeutic agent modulates NK3 protein activity by at least about 0.01%, at least about 0.1%, at least about 1%, at least about 2%, at least about 3%, at least about 4%, at least about 5%, at least about 10%, at least about 20%, at least about 30%, at least about 40%, or at least about 50% relative to NK3 protein activity in a standard.

In some embodiments, the therapeutic agent modulates expression of a TACR3 gene. In some embodiments, the therapeutic agent inhibits expression of a TACR3 gene. In some embodiments, the therapeutic agent downregulates expression of a TACR3 gene. In some embodiments, the therapeutic agent increases expression of a TACR3 gene. In some embodiments, the therapeutic agent upregulates expression of a TACR3 gene.

In some embodiments, administration of the therapeutic agent selectively modulates activity of the NK3 protein or expression of the TACR3 gene in peripheral tissues relative to the central nervous system. In some embodiments, administration of the therapeutic agent selectively modulates activity of the NK3 protein or expression of the TACR3 gene in the peripheral nervous system relative to the central nervous system. In some embodiments, administration of the therapeutic agent does not cause one or more pharmacological effects associated with NK3 modulation in the CNS. In some embodiments, administration of the therapeutic agent does not cause one or more pharmacological effects selected from thermoregulatory dysfunction, bone mineral density, paresthesia, thrombophlebitis, and change in gonadotropin levels (e.g., luteinizing hormone).

In some embodiments, the therapeutic agent is restricted to peripheral tissues. In some embodiments, the therapeutic agent is restricted to the peripheral nervous system. In some embodiments, the therapeutic agent does not substantially cross the blood brain barrier. In some embodiments, administration of the therapeutic agent results in a ratio of blood plasma concentration to brain tissue concentration in the subject of, for example, at least about 2:1, at least about 3:1, at least about 4:1, at least about 5:1, at least about 10:1, at least about 15:1, at least about 20:1, at least about 25:1, at least about 50:1 at least about 75:1, at least about 100:1, at least about 150:1, at least about 200:1, at least about 250:1, at least about 300:1, at least about 350:1, at least about 400:1, at least about 450:1, at least about 500:1, or at least about 1000:1. In some embodiments, administration of the therapeutic agent results in a ratio of blood plasma concentration to brain tissue concentration in the subject of at least about 10:1. In further embodiments, the administration of the therapeutic agent results in a ratio of blood plasma concentration to brain tissue concentration in the subject of at least about 100:1.

In some embodiments, administration of the therapeutic agent results in a ratio of blood plasma concentration to brain tissue concentration in the subject of, for example, from about 2:1 to about 1000:1, from about 2:1 to about 500:1, from about 2:1 to about 250:1, from about 2:1 to about 100:1, from about 2:1 to about 75:1, from about 2:1 to about 50:1, from about 2:1 to about 25:1, from about 2:1 to about 20:1, from about 2:1 to about 15:1, from about 2:1 to about 10:1, from about 2:1 to about 5:1, from about 2:1 to about 4:1, or from about 2:1 to about 3:1. In some embodiments, administration of the therapeutic agent results in a ratio of blood plasma concentration to brain tissue concentration in the subject of, for example, from about 4:1 to about 1000:1, from about 4:1 to about 500:1, from about 4:1 to about 250:1, from about 4:1 to about 100:1, from about 4:1 to about 75:1, from about 4:1 to about 50:1, from about 4:1 to about 25:1, from about 4:1 to about 20:1, from about 4:1 to about 15:1, from about 4:1 to about 10:1, or from about 4:1 to about 5:1. In some embodiments, administration of the therapeutic agent results in a ratio of blood plasma concentration to brain tissue concentration in the subject of, for example, from about 10:1 to about 1000:1, from about 10:1 to about 500:1, from about 10:1 to about 250:1, from about 10:1 to about 100:1, from about 10:1 to about 75:1, from about 10:1 to about 50:1, from about 10:1 to about 25:1, from about 10:1 to about 20:1, or from about 10:1 to about 15:1. In some embodiments, administration of the therapeutic agent results in a ratio of blood plasma concentration to brain tissue concentration in the subject of, for example, from about 2:1 to about 20:1.

In some embodiments, the therapeutic agent contemplated herein is a small molecule, a protein, or a nucleic acid. In some embodiments, the therapeutic agent is a small molecule. Non-limiting examples of small molecule therapeutic agents include those disclosed in EP0673928; WO1995032948; WO1997010211; WO1997019928; WO1997019927; WO1997021680; WO1998018785; WO1998057972; WO1999026924; WO1999001451; WO2000021931; WO2000064877; WO2000039114; WO2002013825; WO2002094821; WO2002083645; WO2002038547; WO2002038548; WO2002044154; WO2002043734; WO2002044165; WO2004066951; WO2004066950; WO2005014575; WO2005061462; WO2005016884; WO2005110987; US2005148601; WO2006050989; WO2006050991; WO2006050992; WO2006130080; WO2006137789; WO2007086799; WO2007018465; WO2007018469; WO200700396; WO2007035157; WO2007018466; WO2007035156; WO2007035158; WO2007069977; WO2007012900; WO2007028654; US20070219214; EP1829556; WO2008115140; WO2008131779; WO2008148688; WO2008148689; WO2008151969; WO2008081012; WO2009130240; WO2009156339; WO2009033995; WO2009019163; WO2009024502; WO2009072643; WO2010106081; WO2010045948; WO2010028655; WO2010086259; WO2010015626; WO2020101017; JP201081888; WO2011110183; WO2011121137; WO2012039718; WO2012009227; WO2012004207; WO2013050424; WO2014154895; WO2014154896; WO2014089019; WO2015200594; WO2015033163; WO2019210327; WO2019074081; and WO2021030335.

In some embodiments, the small molecule therapeutic agent is selected from fezolinetant, pavinetant, talnetant, osanetant, elinzanetant, senktide, GSK172981, GSK256471, SB222200, SB235375, SJX-653, MLE-301, SB218795, GR138676, GR138678, CAM-2425, SSR241586, SSR146977, PD154740, PD157672, PD163416, PD160946, PD161182, SCH206272, GSK 256471, SB48968. In some embodiments, the small molecule therapeutic agent is selected from fezolinetant, pavinetant, talnetant, osanetant, elinzanetant, senktide, GSK172981, GSK256471, SB222200, SB235375, SJX-653, MLE-301, SB218795, GR138676, GR138678, CAM-2425, SSR241586, SSR146977, PD154740, PD157672, PD163416, PD160946, and PD161182. In some embodiments, the small molecule therapeutic agent is selected from fezolinetant, pavinetant, talnetant, osanetant, elinzanetant, GSK172981, GSK256471, SB222200, SB235375, SJX-653. In some embodiments, the small molecule therapeutic agent is selected from 2-diphenylquinoline-4-carbohydrazide, PD161182, SR142801, CS-003, and MLE-301.

In some embodiments, the therapeutic agent is a protein. In some embodiments, the protein therapeutic agent is selected from antibodies, enzymes, hormonal proteins, signaling proteins, and fusion proteins, or functional fragments thereof. In some embodiments, the therapeutic agent is an antibody, or a functional fragment thereof, that selectively binds NK3 or neurokinin B. Non-limiting examples of antibodies or functional fragments thereof that selectively bind NK3 include those disclosed in WO2012020162A1. Non-limiting examples of antibodies or functional fragments thereof that selectively bind neurokinin B include those disclosed in WO2003102136 or WO2020101017. In some embodiments, the therapeutic agent is a fusion protein that selectively binds NK3 or neurokinin B. In some embodiments, the protein therapeutic agent is an NK3 activator. Non-limiting examples of protein therapeutic agents that activate NK3 are selected from neurokinin B, neurokinin A, hemokinin 1, Substance P, [Pro⁷] neurokinin B, [MePhe⁷]-neurokinin B and its derivatives (see Misu et al., Med. Chem. 2014, 57, 20, 8646-8651), senktide and its derivatives (see Misu et al., Med. Chem. 2014, 57, 20, 8646-8651), kassinin, eledoisin, NK3R-A1, NK3R-A2, Senktide analogue, and Scyliorhinin II. In other embodiments, the protein therapeutic agent is an NK3 inhibitor. Non-limiting examples of the protein therapeutic agent that activate NK3 are selected from [Trp⁷, β-Ala⁸] neurokinin A, and [MePhe⁷ β-Ala⁸] neurokinin A (see Drapeau et al., Regulatory Peptides, Volume 31, Issue 2, 15 Nov. 1990), FK 224, GR138676, Substance P (1-7) (Arg-Pro-Lys-Pro-Gln-Gln-Phe), and GR 94800 TFA (Bz-AA-{D-Trp}-F-{D-Pro}-P-{Nle}-NH₂).

In further embodiments, the protein therapeutic agent comprises one or more CDRs from the following antibodies: NB300-102 and NBP1-82561 from Novus Biologicals, ABIN500361 and ABIN725735 from antibodies-online, GTX31316 and GTX70489 from GeneTex, A04916 and A30848 from Boster Biological Technology, PAB13001 and PAB27695 from Abnova Corporation, TA306441 and AP01273PU-N from OriGene, LS-C34712 and LS-C120754 from LifeSpan BioSciences, Inc., PA5-20392 and PA5-102108 from Invitrogen Antibodies, bs-0166R and bs-0914R from Bioss, orb74850 and orb339175 from Biorbyt, AP30602PU-N and AP05734PU-N from Acris Antibodies GmbH, CSB-PA003454 and CSB-PA023070ESR2HU from Cusabio Biotech Co., Ltd, OAAF05000 and ARP64678_P050 from Aviva Systems Biology, APS10550 and AP14222b from Abgent, DF4997 from Affinity Biosciences, 4255 from ProSci, FNab08472 from Wuhan Fine Biotech Co., Ltd., BS2736 from Bioworld Technology, Inc, 22334-1-AP from Proteintech Group, 115972 and 129584 from NovoPro Bioscience Inc., STJ29300 and STJ94498 from St John's Laboratory, ABP51965 and ABP57531 from Abbkine Scientific Co. Ltd., SAB3500368 and HPA009418 from MilliporeSigma/Merck KGaA, MBS150256 and MBS9126076 from MyBioSource, HPA009418 from Atlas Antibodies, 250776 and 250777 from Abbiotec, 868901 and 868902 from BioLegend, abx217136 and abx006440 from Abbexa, AMM06701G and APR13683G from Leading Biology, 224546 and 224546-AP from United States Biological, CPBT-66754RT and DPABH-02783 from Creative Diagnostics, 119-10140 and 119-10172 from RayBiotech, Inc., 18-461-10149 and 18-461-10183 from GenWay, and 600-401-DC1 from Rockland Immunochemicals, Inc.

In other embodiments, the therapeutic agent is a nucleic acid, such as an RNA molecule, such as an interfering RNA (RNAi) molecule. RNA interference is a biological process in which RNA molecules are involved in sequence-specific suppression of gene expression by double-stranded RNA, through translational or transcriptional repression. In some embodiments, the therapeutic agent is an shRNA, an siRNA, a microRNA, an asymmetric interfering RNA that is complementary to a portion of human TACR3 gene (NCBI Gene ID: 6870), or any combination thereof. In some embodiments, the therapeutic agent is an antisense molecule, a lhRNA, an miRNA embedded shRNA, a small internally segmented RNA, that is complementary to a portion of human TACR3 gene (NCBI Gene ID: 6870) or any combination thereof. In some embodiments, the therapeutic agent is administered via a viral vector.

### Pharmaceutical Formulations

In some aspects, the present disclosure provides a pharmaceutical composition comprising an NK3 modulator and at least one pharmaceutically acceptable excipient.

An NK3 modulator may be formulated in any suitable pharmaceutical formulation. A pharmaceutical formulation of the present disclosure typically contains an active ingredient (e.g., an NK3 modulator), and one or more pharmaceutically acceptable excipients or carriers, including but not limited to: inert solid diluents and fillers, diluents, sterile aqueous solution and various organic solvents, permeation enhancers, antioxidants, solubilizers, and adjuvants. Preparations for such pharmaceutical composition are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000; Martindale, The Extra Pharmacopoeia, Thirty-Second Edition (The Pharmaceutical Press, London, 1999).

Methods for formulation of the therapeutic proteins can include formulating any of the therapeutic proteins of the present disclosure with one or more inert, pharmaceutically-acceptable excipients or carriers to form semi-solid or liquid composition. These pharmaceutical compositions can comprise at least one active ingredient (e.g., a therapeutic agent of the present disclosure). Such compositions can further contain nontoxic, auxiliary substances, for example, solvents, buffers, or emulsifying agents, pH buffering agents, and other pharmaceutically-acceptable additives. Alternatively, a therapeutic protein of the present disclosure can be lyophilized or in powder form for re-constitution with a suitable vehicle, e.g., sterile pyrogen-free water, before use.

The amount of a therapeutic agent of the present disclosure will be dependent on the mammal being treated, the severity of the disorder or condition, the rate of administration, the disposition of the therapeutic protein and the discretion of the prescribing physician.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

The following examples are illustrative and non-limiting to the scope of the compositions, devices, uses, and methods disclosed herein.

### Example 1: In Vivo Migraine Headache Model

Surgeries were performed as previously described in Oshinsky, M. L. & Gomonchareonsiri, S., Episodic dural stimulation in awake rats: a model for recurrent headache, 47 HEADACHE 1026-1036 (2007). Adult male Sprague-Dawley rats were habituated towards handling for 1-2 weeks before undergoing dural cannulation surgery under isoflurane anesthesia. Attention was given not to disturb the dura. A custom flange guide cannula (22GA, Plastics One) was inserted. The cannula was fixed to the bone with small screws and dental cement.

After 1-2 weeks of recovery from surgery, animals received supradural infusions of an inflammatory soup comprising 2 mM histamine, bradykinin, serotonin, 0.2 mM prostaglandin E2 under brief isoflurane anesthesia (1.5-3%) for 5 consecutive days (1/day) (i.e., the 5-day sensitization period). If rats showed no signs of facial sensitivity after the final infusion they were removed from the study.

After the 5-day sensitization period, rats had a one-week wash-out period before nitroglycerin challenge (GTN, 0.1 mg/kg, I.P) and either fezolinetant (30 or 100 mg/kg, PO), SB 235375 (30 or 100 mg/kg, PO), or vehicle (0.5% methylcellulose in sterile water for fezolinetant, sterile water for SB 235375, PO). The design was within-subjects with the same animals receiving multiple treatments in a crossover fashion with at least one week washout between each treatment. Evoked sensory testing for mechanical allodynia was done by von Frey periorbital threshold testing. Sensory testing occurred during the day using von Frey filaments with reproducible calibrated buckling forces varying from 0.4-10 g and utilizing the up-and-down method. See Dixon, W. J., The Up-and-Down Method for Small Samples, 60 J. AM. STAT. ASSOC., 967-978 (1965); Chaplan, S. R., Bach, F. W., Pogrel, J. W., Chung, J. M. & Yaksh, T. L., Quantitative assessment of tactile allodynia in the rat paw, 53 J. NEUROSCI. METHODS 55-63 (1994) (hereinafter “Chaplan 1994”). Based on the response pattern and the force of the final filament, the periorbital threshold (g) was calculated according to the prior art method set forth, e.g., in Chaplan 1994 and Bonin, R. P., Bories, C. & De Koninck, Y., A simplified up-down method (SUDO) for measuring mechanical nociception in rodents using von Frey filaments, 10 MOL. PAIN 26 (2014). A blinded investigator tested periorbital sensitivity before drug administration, and 45 min and 1.5 h after drug administration. Studies were conducted with n=13-22.

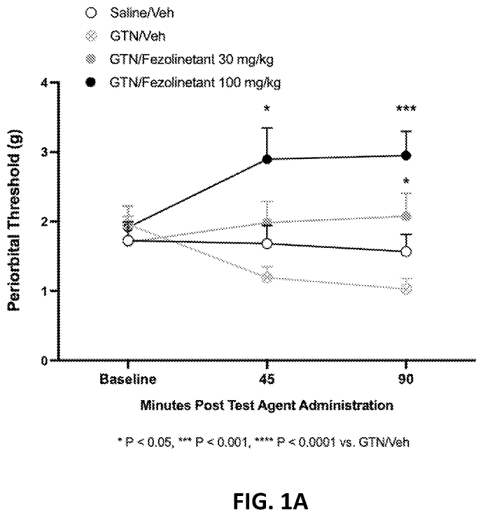

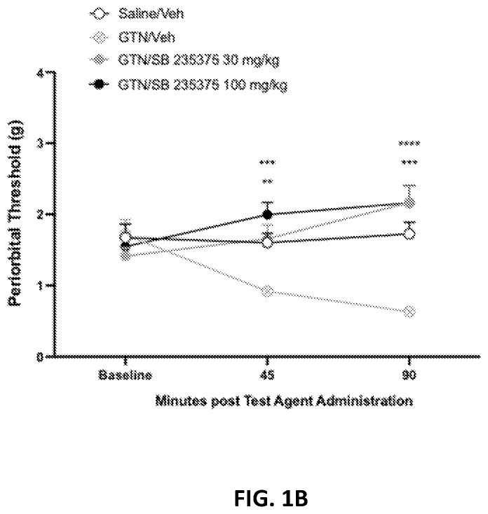

The results of the in vivo migraine headache model are provided in FIGS. 1A-B. Specifically, FIGS. 1A-B show the effects of a brain penetrant NK3 antagonist, fezolinetant, (FIG. 1A) and a peripherally restricted NK3 antagonist, SB 235375, (FIG. 1B) on periorbital threshold following GTN exposure in rats with inflammatory soup-sensitized trigeminal ganglia. Baseline withdrawal thresholds between groups were not significantly different (baseline measurements from FIGS. 1A-B). As shown in FIG. 1A, withdrawal thresholds in rats receiving GTN and 30 mg/kg fezolinetant were significantly higher at the 90 min timepoint than those receiving either control group (saline+vehicle or GTN+vehicle). Withdrawal thresholds in rats receiving GTN and 100 mg/kg fezolinetant were significantly higher at both timepoints compared to each control group, demonstrating an NK3-dependent analgesic effect at both doses (2 way ANOVA, vs GTN/veh). Further, FIG. 1A shows dose-dependent increase in periorbital threshold values for fezolinetant treatment, as the 100 mg/kg dose of fezolinetant resulted in a higher withdrawal threshold than the 30 mg/kg dose of fezolinetant.

FIG. 1B shows the effectiveness of SB 235375, NK3 antagonist with minimal brain penetration, in the same sensitized in vivo migraine headache model. See Hay, D. W. P. et al., Nonpeptide tachykinin receptor antagonists. III. SB 235375, a low central nervous system-penetrant, potent and selective neurokinin-3 receptor antagonist, inhibits citric acid-induced cough and airways hyper-reactivity in guinea pigs, 300 J. PHARMACOL. EXP. THER. 314-323 (2002). Similar to those treated with fezolinetant, rats co-administered GTN and SB 235375 exhibited higher pain thresholds compared to GTN and vehicle-treated rats at both doses (30 and 100 mg/kg) and time points (45, 90 minutes) (see FIG. 1B). Thus, as shown in FIGS. 1A-B, both NK3 antagonists, including the CNS-penetrant fezolinetant and the peripherally restricted SB 235375, demonstrate efficacy in treating migraine.

### Example 2: Luteinizing Hormone (LH) Inhibition Studies

Studies measuring LH were performed on castrated male Sprague-Dawley rats. Briefly, animals were castrated through a midline scrotal incision using Zoletil® 50 as an anesthetic. Surgeries were performed at least 15 days before compound testing.

Serum was collected at baseline, then every 15 minutes starting from 30 minutes after PO (periorbital) dosing with vehicle, fezolinetant (30 mg/kg), or SB 235375 (30 and 100 mg/kg). Blood was collected from the jugular vein, transferred into 1.5-mL centrifuge tubes, and centrifuged at 8000 g for 15 minutes to separate the serum. Serum LH levels were determined using a rat luteinizing hormone ELISA kit (Colorimetric, Novus). The assays were performed following the manufacturer's protocol. Group sizes were from n=10 for the vehicle and SB 235375 groups and n=4 for fezolinetant.

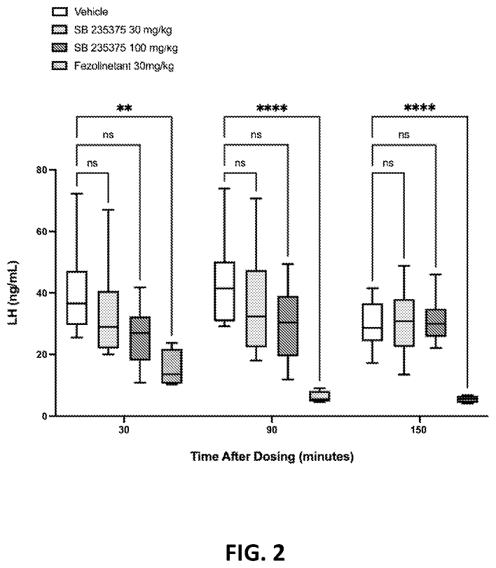

The results of the LH inhibition studies are provided in FIG. 2. The data shown in FIG. 2 confirm that brain exposures from SB 235375 at the doses utilized to treat migraine would be inadequate to elicit a substantial CNS-dependent effect. Both fezolinetant (30 mg/kg) and SB 235375 (30 and 100 mg/kg) were tested for their capacity to suppress the high LH levels reported in castrated rats. See Depypere 2021; Modi, M. & Dhillo, W. S., Neurokinin 3 Receptor Antagonism: A Novel Treatment for Menopausal Hot Flushes, 109 NEUROENDOCRINOLOGY, 242-248 (2019). Rats treated with fezolinetant (30 mg/kg) showed significantly reduced LH serum concentrations at all measured time points (30, 90, and 150 min). In contrast, SB 235375-treated animals did not exhibit a significant difference in LH levels compared to the vehicle-treated group (see FIG. 2).

The mean LH values underlying the data provided in FIG. 2 are provided below, in Table 1.

TABLE 1

| Time | | SB 235375 | | SB 235375 | | fezolinetant | |
|---|---|---|---|---|---|---|---|
| (min) | Vehicle | 30 mg/kg | | 100 mg/kg | | 30 mg/kg | |
| 0 | 34.4 ng/ml | 31.6 | ng/ml | 34.7 | ng/ml | 37.3 | ng/ml |
| 30 | 40.1 ng/ml | 33.4 | ng/ml | 25.7 | ng/ml | 15.2 | ng/ml |
| 90 | 42.7 ng/ml | 35.8 | ng/ml | 29.7 | ng/ml | 6.1 | ng/ml |
| 150 | 29.3 ng/ml | 30.4 | ng/ml | 31.1 | ng/mL | 5.5 | ng/ml |

As shown in Table 1, 150 minutes after administration, the LH values for vehicle, 30 mg/kg dose of SB 235375, and 100 mg/kg dose of SB 235375 were essentially equivalent (29.3 ng/ml, 30.4 ng/mL, and 31.1 ng/mL, respectively), while fezolinetant caused a significant reduction in LH levels (5.5 ng/mL). Thus, while fezolinetant caused a reduction in LH levels of roughly 85% compared to vehicle treatment, minimal changes were observed at the tested doses of SB 235375 150 minutes following treatment (roughly 4% and 10% reductions for the 30 and 100 mg/kg doses, respectively). Similarly, at 30- and 90-minutes post-treatment, fezolinetant caused a much more significant reduction in LH levels (approximately 60% and 85% reduction compared to vehicle, respectively) compared to the same 30 mg/kg dose of SB 235375 (approximate increases of +4% and +13%, respectively) and 100 mg/kg dose of SB 235375 (roughly 26% and 14% reductions, respectively).

The forgoing data presented in FIGS. 1A-B and in FIG. 2 demonstrate that NK3 modulators are effective in the treatment of headaches (e.g., migraine headaches), and that this efficacy is retained when the NK3 modulator is peripherally restricted (e.g., does not accumulate to a clinically significant concentration in the brain). Further, the data show that use of a peripherally restricted NK3 modulator provides the additional advantage of avoiding unwanted side effects associated with NK3 modulation in the CNS (e.g., changes in gonadotropin levels such as LH). The foregoing examples are merely exemplary and are not intended to limit the scope of the present disclosure.

What is claimed is:

1. A method for treating or preventing a disease or condition in a subject in need thereof, the method comprising administering to the subject a therapeutically effective amount of a therapeutic agent that modulates activity of a Neurokinin Receptor 3 (“NK3”) protein or modulates expression of a TACR3 gene, wherein the disease or condition is selected from the group consisting of: migraine, medication overuse headache, cluster headache, general headache, trigeminal neuralgia, and orofacial pain.

2. The method of claim 1, wherein the disease or condition is selected from the group consisting of: migraine, medication overuse headache, cluster headache, and general headache.

3. The method of claim 1, wherein the disease or condition is migraine.

4. The method of any one of claims 1 to 3, wherein the therapeutic agent modulates activity of a NK3 protein.

5. The method of claim 4, wherein the therapeutic agent is a NK3 antagonist.

6. The method of any one of claims 1 to 3, wherein the therapeutic agent modulates expression of a TACR3 gene.

7. The method of claim 6, wherein the therapeutic agent decreases expression of a TACR3 gene.

8. The method of any one of claims 1 to 7, wherein the administration of the therapeutically effective amount of the therapeutic agent does not reduce luteinizing hormone in the subject.

9. The method of any one of claims 1 to 8, wherein the administration of the therapeutic agent results in a ratio of blood plasma concentration to brain tissue concentration of at least 10 to 1 in the subject.

10. The method of claim 9, wherein the administration of the therapeutic agent results in a ratio of blood plasma concentration to brain tissue concentration of at least 100 to 1 in the subject.

11. The method of any one of claims 1 to 10, wherein the administration of the therapeutic agent modulates activity of the NK3 protein or expression of the TACR3 gene in peripheral tissues.

12. The method of claim 11, wherein the administration of the therapeutic agent selectively modulates activity of the NK3 protein or expression of the TACR3 gene in peripheral tissues as compared to in the central nervous system.

13. The method of any one of claims 1 to 12, wherein the therapeutic agent is a small molecule, a protein, or a nucleic acid.

14. The method of claim 13, wherein the therapeutic agent is a small molecule.

15. The method of claim 14, wherein the therapeutic agent is disclosed in EP0673928; WO1995032948; WO1997010211; WO1997019928; WO1997019927; WO1997021680; WO1998018785; WO1998057972; WO1999026924; WO1999001451; WO2000021931; WO2000064877; WO2000039114; WO2002013825; WO2002094821; WO2002083645; WO2002038547; WO2002038548; WO2002044154; WO2002043734; WO2002044165; WO2004066951; WO2004066950; WO2005014575; WO2005061462; WO2005016884; WO2005110987; US2005148601; WO2006050989; WO2006050991; WO2006050992; WO2006130080; WO2006137789; WO2007086799; WO2007018465; WO2007018469; WO200700396; WO2007035157; WO2007018466; WO2007035156; WO2007035158; WO2007069977; WO2007012900; WO2007028654; US20070219214; EP1829556; WO2008115140; WO2008131779; WO2008148688; WO2008148689; WO2008151969; WO2008081012; WO2009130240; WO2009156339; WO2009033995; WO2009019163; WO2009024502; WO2009072643; WO2010106081; WO2010045948; WO2010028655; WO2010086259; WO2010015626; WO2020101017; JP201081888; WO2011110183; WO2011121137; WO2012039718; WO2012009227; WO2012004207; WO2013050424; WO2014154895; WO2014154896; WO2014089019; WO2015200594; WO2015033163; WO2019210327; WO2019074081; or WO2021030335.

16. The method of claim 14, wherein the small molecule is selected from fezolinetant, pavinetant, talnetant, osanetant, elinzanetant, senktide, GSK172981, GSK256471, SB222200, SB235375, SJX-653, MLE-301, SB218795, GR138676, GR138678, CAM-2425, SSR241586, SSR146977, PD154740, PD157672, PD163416, PD160946, PD161182, SCH206272, GSK 256471, and SB48968.

17. The method of claim 16, wherein the small molecule is selected from fezolinetant, pavinetant, talnetant, osanetant, elinzanetant, senktide, GSK172981, GSK256471, SB222200, SB235375, SJX-653, MLE-301, SB218795, GR138676, GR138678, CAM-2425, SSR241586, SSR146977, PD154740, PD157672, PD163416, PD160946, and PD161182.

18. The method of claim 16, wherein the small molecule is selected from fezolinetant, pavinetant, talnetant, osanetant, elinzanetant, GSK172981, GSK256471, SB222200, SB235375, SJX-653, and MLE-301.

19. The method of claim 13, wherein the therapeutic agent is a protein.

20. The method of claim 19, wherein the therapeutic agent is an antibody or a functional fragment thereof that selectively binds NK3 or neurokinin B.

21. The method of claim 20, wherein the therapeutic agent is an antibody or a functional fragment thereof that selectively binds NK3 disclosed in WO2012020162A1.

22. The method of claim 20, wherein the therapeutic agent is an antibody or a functional fragment thereof that selectively binds neurokinin B disclosed in WO2003102136 or WO2020101017.

23. The method of claim 19, wherein the therapeutic agent is a fusion protein.

24. The method of claim 13, wherein the therapeutic agent is a nucleic acid.

25. The method of claim 24, wherein the therapeutic agent is an RNAi molecule.

26. The method of claim 25, wherein the RNAi molecule is an shRNA, an siRNA, a microRNA, or an asymmetric interfering RNA.

27. The method of claim 24, wherein the therapeutic agent is an antisense molecule, a lhRNA, an miRNA embedded shRNA, or a small internally segmented RNA.

28. The method of any one of claims 1 to 27, wherein the therapeutic agent is administered via a viral vector.

29. The method of any one of claims 1 to 28, wherein the subject is a human.

30. An NK3 modulator for use in the treatment or prevention of a disease or condition in a subject in need thereof, wherein the disease or condition is selected from the group consisting of: migraine, cluster headache, general headache, trigeminal neuralgia, and orofacial pain; and wherein the NK3 modulator is a therapeutic agent that modulates activity of an NK3 protein or modulates expression of a TACR3 gene.

31. The NK3 modulator for use of claim 30, wherein the disease or condition is selected from the group consisting of: migraine, medication overuse headache, cluster headache, and general headache.

32. The NK3 modulator for use of claim 30 or claim 31, wherein the disease or condition is migraine.

33. The NK3 modulator for use of any one of claims 30 to 32, wherein the NK3 modulator modulates activity of an NK3 protein.

34. The NK3 modulator for use of any one of claims 30 to 33, wherein NK3 modulator is an NK3 antagonist.

35. The NK3 modulator for use of any one of claims 30 to 34, wherein the NK3 modulator modulates expression of a TACR3 gene.

36. The NK3 modulator for use of any one of claims 30 to 35, wherein the therapeutic agent decreases expression of a TACR3 gene.

37. The NK3 modulator for use of any one of claims 30 to 36, wherein administration of the therapeutically effective amount of the NK3 modulator to the subject does not reduce luteinizing hormone in the subject.

38. The NK3 modulator for use of any one of claims 30 to 37, wherein administration of the NK3 modulator to the subject results in a ratio of blood plasma concentration to brain tissue concentration of at least 10 to 1 in the subject.

39. The NK3 modulator for use of claim 38, wherein administration of the NK3 modulator to the subject results in a ratio of blood plasma concentration to brain tissue concentration of at least 100 to 1 in the subject.

40. The NK3 modulator for use of any one of claims 30 to 39, wherein the NK3 modulator modulates activity of the NK3 protein or expression of the TACR3 gene in peripheral tissues.

41. The NK3 modulator for use of claim 40, wherein the NK3 modulator selectively modulates activity of the NK3 protein or expression of the TACR3 gene in peripheral tissues as compared to in the central nervous system.

42. The NK3 modulator for use of any one of claims 30 to 41, wherein the NK3 modulator is a small molecule, a protein, or a nucleic acid.

43. The NK3 modulator for use of any one of claims 30 to 42, wherein the NK3 modulator is a small molecule.

44. The NK3 modulator for use of claim 43, wherein the small molecule is disclosed in EP0673928; WO1995032948; WO1997010211; WO1997019928; WO1997019927; WO1997021680; WO1998018785; WO1998057972; WO1999026924; WO1999001451; WO2000021931; WO2000064877; WO2000039114; WO2002013825; WO2002094821; WO2002083645; WO2002038547; WO2002038548; WO2002044154; WO2002043734; WO2002044165; WO2004066951; WO2004066950; WO2005014575; WO2005061462; WO2005016884; WO2005110987; US2005148601; WO2006050989; WO2006050991; WO2006050992; WO2006130080; WO2006137789; WO2007086799; WO2007018465; WO2007018469; WO200700396; WO2007035157; WO2007018466; WO2007035156; WO2007035158; WO2007069977; WO2007012900; WO2007028654; US20070219214; EP1829556; WO2008115140; WO2008131779; WO2008148688; WO2008148689; WO2008151969; WO2008081012; WO2009130240; WO2009156339; WO2009033995; WO2009019163; WO2009024502; WO2009072643; WO2010106081; WO2010045948; WO2010028655; WO2010086259; WO2010015626; WO2020101017; JP201081888; WO2011110183; WO2011121137; WO2012039718; WO2012009227; WO2012004207; WO2013050424; WO2014154895; WO2014154896; WO2014089019; WO2015200594; WO2015033163; WO2019210327; WO2019074081; or WO2021030335.

45. The NK3 modulator for use of claim 43 or claim 44, wherein the small molecule is selected from fezolinetant, pavinetant, talnetant, osanetant, elinzanetant, senktide, GSK172981, GSK256471, SB222200, SB235375, SJX-653, MLE-301, SB218795, GR138676, GR138678, CAM-2425, SSR241586, SSR146977, PD154740, PD157672, PD163416, PD160946, PD161182, SCH206272, GSK 256471, and SB48968.

46. The NK3 modulator for use of any one of claims 43 to 45, wherein the small molecule is selected from fezolinetant, pavinetant, talnetant, osanetant, elinzanetant, senktide, GSK172981, GSK256471, SB222200, SB235375, SJX-653, MLE-301, SB218795, GR138676, GR138678, CAM-2425, SSR241586, SSR146977, PD154740, PD157672, PD163416, PD160946, and PD161182.

47. The NK3 modulator for use of any one of claims 43 to 46, wherein the small molecule is selected from fezolinetant, pavinetant, talnetant, osanetant, elinzanetant, GSK172981, GSK256471, SB222200, SB235375, SJX-653, and MLE-301.

48. The NK3 modulator for use of claim 42, wherein the NK3 modulator is a protein.

49. The NK3 modulator for use of claim 48, wherein the NK3 modulator is an antibody or a functional fragment thereof that selectively binds NK3 or neurokinin B.

50. The NK3 modulator for use of claim 49, wherein the NK3 modulator is an antibody or a functional fragment thereof that selectively binds NK3 disclosed in WO2012020162A1

51. The NK3 modulator for use of claim 50, wherein the NK3 modulator is an antibody or a functional fragment thereof that selectively binds neurokinin B disclosed in WO2003102136 or WO2020101017.

52. The NK3 modulator for use of claim 48, wherein the NK3 modulator is a fusion protein.

53. The NK3 modulator for use of claim 42, wherein the NK3 modulator is a nucleic acid.

54. The NK3 modulator for use of claim 53, wherein the NK3 modulator is an RNAi molecule.

55. The NK3 modulator for use of claim 54, wherein the RNAi molecule is an shRNA, an siRNA, a microRNA, or an asymmetric interfering RNA.

56. The NK3 modulator for use of claim 55, wherein the NK3 modulator is an antisense molecule, a lhRNA, an miRNA embedded shRNA, or a small internally segmented RNA.

57. The NK3 modulator for use of any one of claims 30 to 56, wherein the NK3 modulator is administered via a viral vector.

58. The NK3 modulator for use of any one of claims 30 to 57, wherein the subject is a human.
